(19) United States
(12) Patent Application Publication
(10) Pub. No.: US 2025/0258129 A1
(43) Pub. Date: Aug. 14, 2025

(54) GAS SENSOR AND CONCENTRATION MEASUREMENT METHOD USING GAS SENSOR

(71) Applicant: NGK INSULATORS, LTD., Nagoya-shi (JP)

(72) Inventors: Yusuke WATANABE, Nagoya-shi (JP); Daichi ICHIKAWA, Nagoya-shi (JP)

(21) Appl. No.: 19/098,027

(22) Filed: Apr. 2, 2025

Related U.S. Application Data
(63) Continuation of application No. PCT/JP2023/029128, filed on Aug. 9, 2023.

(30) Foreign Application Priority Data
Oct. 6, 2022 (JP) 2022-161631

(51) Int. Cl. G01N 27/407; G01N 27/60; G01N 33/00

(52) U.S. Cl. CPC G01N 27/407; G01N 27/605; G01N 33/0022; G01N 33/004

(57) ABSTRACT

A sensor element includes first to third chambers communicating sequentially from a gas inlet, an adjustment pump cell pumps oxygen into the first chamber so that an H/C component is oxidized, a first measurement pump cell pumps out oxygen from the second chamber so that all H₂O and CO₂ contained in the measurement gas are reduced, a second measurement pump cell pumps oxygen into the third chamber to selectively oxidize H₂ generated by reduction, a concentration of H₂O is identified from values of currents generated by pumping-in by the adjustment pump cell and the second measurement pump cell, and a concentration of CO₂ is identified based on the identified concentration of H₂O, the value of the current generated by pumping-in by the adjustment pump cell, and a value of a current generated by pumping-out by the first measurement pump cell.

### CROSS-REFERENCE TO RELATED APPLICATION

This application is a continuation application of PCT/JP2023/029128, filed on Aug. 9, 2023, which claims the benefit of priority of Japanese Patent Application No. JP2022-161631, filed on Oct. 6, 2022, the entire contents of which are incorporated herein by reference.

### BACKGROUND

### Technical Field

The present invention relates to a multi-gas sensor capable of sensing a plurality of types of sensing target gas components and measuring concentrations thereof.

### Description of the Background Art

In measurement for managing the amount of an emitted exhaust gas from a vehicle, technology of measuring concentrations of water vapor (H₂O) and carbon dioxide (CO₂) has already been known (see Japanese Patent No. 5918177, No. 6469464, and No. 6469462, for example). In each of gas sensors disclosed in Japanese Patent No. 5918177 and No. 6469464, a water vapor (H₂O) component and a carbon dioxide (CO₂) component can be measured in parallel. As for a gas sensor disclosed in Japanese Patent No. 6469462, a water vapor (H₂O) component can accurately be measured even when a measurement gas contains carbon dioxide (CO₂).

In the gas sensor disclosed in Japanese Patent No. 5918177 having a three-chamber configuration, firstly, a main pump cell as a pump cell for a first internal space operates to pump out O₂ contained in a measurement gas introduced into the first internal space and to reduce all H₂O and CO₂ similarly contained in the measurement gas once to generate H₂ and CO. The measurement gas containing these H₂ and CO is introduced into a second internal space and further into a third internal space. A first measurement pump cell as a pump cell for the second internal space then pumps in O₂ to selectively oxidize H₂ to generate H₂O, and, further, a second measurement pump cell as a pump cell for the third internal space pumps in O₂ to oxidize CO to generate CO₂. Concentrations of H₂O and CO₂ in the measurement gas are respectively measured based on magnitudes of pump currents flowing through the first measurement pump cell and the second measurement pump cell when H₂ and CO are oxidized.

When the measurement gas has a significant hydrocarbon gas component (H/C component) content as with a rich atmospheric gas, however, an H/C component is oxidized in the second internal space and the third internal space to generate H₂O and CO₂ in the gas sensor disclosed in Japanese Patent No. 5918177, and H₂O and CO₂ as generated are mixed with H₂O and CO₂ contained originally in the measurement gas, so that the concentrations of H₂O and CO₂ sometimes cannot accurately be measured by the above-mentioned scheme.

In the gas sensor disclosed in Japanese Patent No. 6469462 having a two-chamber configuration, for improvement in selective H₂ oxidation property, an alloy of Au and another noble metal (e.g., Pt, Rh, and Ru) is used as a material for a measurement inner pump electrode as an in-space pump electrode forming a measurement pump cell as a pump cell for a second internal space, and an abundance ratio of Au in a surface of the electrode is 25 at % or more.

This material for the electrode is seemingly applicable to a first measurement inner pump electrode of the first measurement pump cell selectively oxidizing H₂ similarly in the gas sensor disclosed in Japanese Patent No. 5918177.

The first measurement inner pump electrode, however, is provided at a location to be at a higher temperature than a second measurement inner pump electrode as an in-space pump electrode forming the second measurement pump cell in the gas sensor disclosed in Japanese Patent No. 5918177, so that use of such a material for the electrode might cause evaporation of Au in the electrode to change sensitivity during a long-term use.

### SUMMARY

The present invention relates to a multi-gas sensor capable of sensing a plurality of types of sensing target gas components and measuring concentrations thereof.

According to the present invention, a gas sensor capable of measuring concentrations of a plurality of sensing target gas components contained in a measurement gas, the measurement gas at least containing water vapor and carbon dioxide, the gas sensor includes: a sensor element having a structure formed of an oxygen-ion conductive solid electrolyte; and a controller controlling operation of the gas sensor, wherein the sensor element includes: a gas inlet through which the measurement gas is introduced; an internal chamber communicating with the gas inlet via a diffusion control part; an adjustment electrode, a first measurement electrode, and a second measurement electrode each provided to face the internal chamber and arranged in order of proximity to the gas inlet while being spaced apart at predetermined intervals; an adjustment pump cell including the adjustment electrode, an out-of-space pump electrode provided at a location other than a location in the internal chamber, and a portion of the solid electrolyte present between the adjustment electrode and the out-of-space pump electrode; a first measurement pump cell including the first measurement electrode, the out-of-space pump electrode, and a portion of the solid electrolyte present between the first measurement electrode and the out-of-space pump electrode; a second measurement pump cell including the second measurement electrode, the out-of-space pump electrode, and a portion of the solid electrolyte present between the second measurement electrode and the out-of-space pump electrode; and a heater heating the sensor element, the adjustment pump cell pumps oxygen into the internal chamber from an external space so that, when the measurement gas having reached the adjustment electrode contains a hydrocarbon gas component, the hydrocarbon gas component is oxidized, the first measurement pump cell pumps oxygen out of the measurement gas having reached the first measurement electrode so that substantially all water vapor and carbon dioxide contained in the measurement gas are reduced, the second measurement pump cell pumps oxygen into the internal chamber to selectively oxidize hydrogen generated by reduction of water vapor and contained in the measurement gas having reached the second measurement electrode, and the controller identifies: a concentration of water vapor contained in the measurement gas based on a value of a hydrocarbon equivalent current as an oxygen pump current flowing between the adjustment electrode and the out-of-space pump electrode when the hydrocarbon gas component is oxidized with oxygen pumped in by the adjustment pump cell and a value of a water vapor equivalent current as an oxygen pump current flowing between the second measurement electrode and the out-of-space pump electrode when hydrogen is oxidized with oxygen pumped in by the second measurement pump cell; and a concentration of carbon dioxide contained in the measurement gas based on the value of the hydrocarbon equivalent current, the value of the water vapor equivalent current, and a value of a total reducing current as an oxygen pump current flowing between the first measurement electrode and the out-of-space pump electrode when water vapor and carbon dioxide are reduced by the first measurement pump cell pumping out oxygen.

Another aspect of the present invention is a concentration measurement method of measuring concentrations of a plurality of sensing target gas components contained in a measurement gas using a gas sensor, the measurement gas at least containing water vapor and carbon dioxide, wherein the gas sensor includes a sensor element having an elongated planar structure formed of an oxygen-ion conductive solid electrolyte, the sensor element includes: a gas inlet through which the measurement gas is introduced; an internal chamber communicating with the gas inlet via a diffusion control part; an adjustment electrode, a first measurement electrode, and a second measurement electrode each provided to face the internal chamber and arranged in order of proximity to the gas inlet while being spaced apart at predetermined intervals; an adjustment pump cell including the adjustment electrode, an out-of-space pump electrode provided at a location other than a location in the internal chamber, and a portion of the solid electrolyte present between the adjustment electrode and the out-of-space pump electrode; a first measurement pump cell including the first measurement electrode, the out-of-space pump electrode, and a portion of the solid electrolyte present between the first measurement electrode and the out-of-space pump electrode; a second measurement pump cell including the second measurement electrode, the out-of-space pump electrode, and a portion of the solid electrolyte present between the second measurement electrode and the out-of-space pump electrode; and a heater heating the sensor element, and the concentration measurement method using the gas sensor includes: a) pumping, using the adjustment pump cell, oxygen into the internal chamber from an external space so that, when the measurement gas having reached the adjustment electrode contains a hydrocarbon gas component, the hydrocarbon gas component is oxidized; b) pumping, using the first measurement pump cell, oxygen out of the measurement gas having reached the first measurement electrode so that substantially all water vapor and carbon dioxide contained in the measurement gas having reached the first measurement electrode are reduced; c) pumping, using the second measurement pump cell, oxygen into the internal chamber to selectively oxidize hydrogen generated by reduction 5 of water vapor and contained in the measurement gas having reached the second measurement electrode; d) identifying a concentration of water vapor contained in the measurement gas based on a value of a hydrocarbon equivalent current as an oxygen pump current flowing between the adjustment electrode and the out-of-space pump electrode when the hydrocarbon gas component is oxidized with oxygen pumped in using the adjustment pump cell and a value of a water vapor equivalent current as an oxygen pump current flowing between the second measurement electrode and the out-of-space pump electrode when hydrogen is oxidized with oxygen pumped in using the second measurement pump cell; and e) identifying a concentration of carbon dioxide contained in the measurement gas based on the value of the hydrocarbon equivalent current, the value of the water vapor equivalent current, and a value of a total reducing current as an oxygen pump current flowing between the first measurement electrode and the out-of-space pump electrode when water vapor and carbon dioxide are reduced by pumping out oxygen using the first measurement pump cell.

According to the present invention, even when a measurement gas containing water vapor and carbon dioxide contains a hydrocarbon gas component as with a rich atmospheric gas, a multi-gas sensor capable of measuring concentrations of water vapor and carbon dioxide while eliminating the influence of the hydrocarbon gas component is implemented.

It is therefore an object of the present invention to provide a multi-gas sensor with more long-term reliability than before capable of simultaneously measuring a water vapor (H₂O) component and a carbon dioxide (CO₂) component, suppressing reduction in measurement accuracy even when a measurement gas has a significant H/C component content, and further being less likely to be subjected to a change in sensitivity during a long-term use.

### BRIEF DESCRIPTION OF THE DRAWINGS

FIG. 1 is a diagram schematically showing one example of a configuration of a gas sensor 100.

FIG. 2 is a block diagram showing functional components implemented by a controller 110.

FIG. 3 is a schematic diagram illustrating entry and exit of gases into and from three chambers comprised in a sensor element 101 of the gas sensor 100.

FIG. 4 is a diagram schematically showing dependence of an oxygen pump current Ip0 on a concentration of an H/C component when a measurement gas contains the H/C component.

FIG. 5 is a diagram showing dependence of an oxygen pump current Ip1 on concentrations of sensing target gas components.

FIG. 6 is a diagram showing dependence of an oxygen pump current Ip2 on concentrations of sensing target gas components.

FIG. 7 is a diagram schematically showing one example of a configuration of a gas sensor 200 according to a modification.

### DESCRIPTION OF THE PREFERRED EMBODIMENTS

### <Configuration of Gas Sensor>

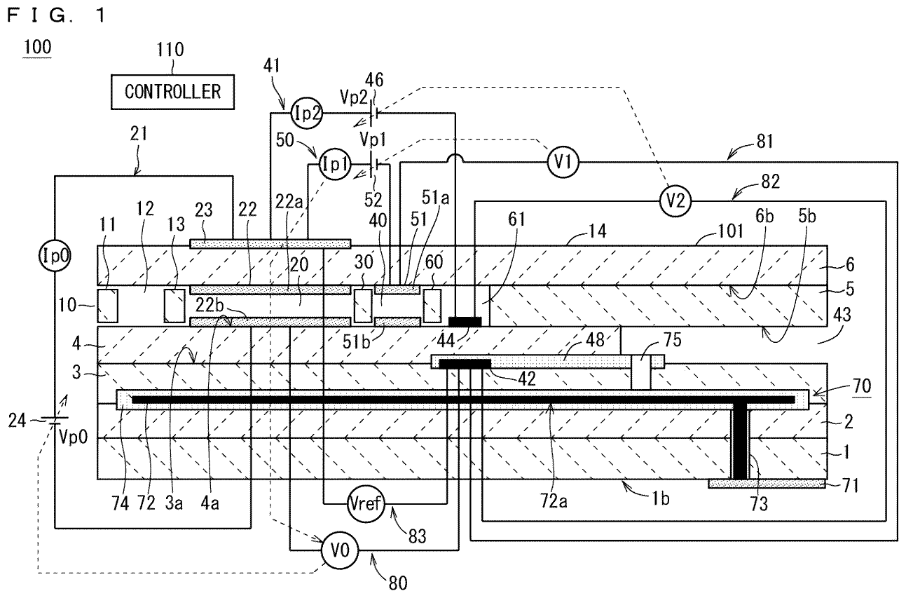

FIG. 1 is a diagram schematically showing one example of a configuration of a gas sensor 100 according to the present embodiment. The gas sensor 100 is a multi-gas sensor sensing a plurality of types of gas components and measuring concentrations thereof using a sensor element 101. Assume that at least water vapor (H₂O) and carbon dioxide (CO₂) are main sensing target gas components of the gas sensor 100 in the present embodiment. The gas sensor 100 is attached to an exhaust path of an internal combustion engine, such as an engine of a vehicle, and is used with an exhaust gas flowing along the exhaust path as a measurement gas, for example. FIG. 1 includes a vertical cross-sectional view taken along a longitudinal direction of the sensor element 101.

The sensor element 101 includes an elongated planar structure (base part) 14 formed of an oxygen-ion conductive solid electrolyte, a first diffusion control part 11 doubling a gas inlet 10 which is formed in one end portion (a left end portion in the figure) of the structure 14 and through which the measurement gas is introduced, and a buffer space 12, a first chamber 20, a second chamber 40, and a third chamber 61 formed in the structure 14 and communicating sequentially from the gas inlet 10 (first diffusion control part 11). The buffer space 12 communicates with the gas inlet 10 (first diffusion control part 11). The first chamber 20 communicates with the buffer space 12 via a second diffusion control part 13. The second chamber 40 communicates with the first chamber 20 via a third diffusion control part 30. The third chamber 61 communicates with the second chamber 40 via a fourth diffusion control part 60.

The structure 14 is formed by laminating a plurality of substrates of ceramics, for example. Specifically, the structure 14 has a configuration in which six layers including a first substrate 1, a second substrate 2, a third substrate 3, a first solid electrolyte layer 4, a spacer layer 5, and a second solid electrolyte layer 6 are sequentially laminated from the bottom. Each layer is formed of an oxygen-ion conductive solid electrolyte, such as zirconia (ZrO₂).

The first diffusion control part 11 doubling as the gas inlet 10, the buffer space 12, the second diffusion control part 13, the first chamber 20, the third diffusion control part 30, the second chamber 40, the fourth diffusion control part 60, and the third chamber 61 are formed in this order between a lower surface 6b of the second solid electrolyte layer 6 and an upper surface 4a of the first solid electrolyte layer 4 on a side of the one end portion of the structure 14. A part extending from the gas inlet 10 to the third chamber 61 is also referred to as a gas distribution part.

The buffer space 12, the first chamber 20, the second chamber 40, and the third chamber 61 are formed to penetrate the spacer layer 5 in a thickness direction. The lower surface 6b of the second solid electrolyte layer 6 is exposed in upper portions in the figure of these chambers and the like, and the upper surface 4a of the first solid electrolyte layer 4 is exposed in lower portions in the figure of these chambers and the like. Side portions of these chambers and the like are each defined by the spacer layer 5 or any of the diffusion control parts. The first chamber 20, the second chamber 40, and the third chamber 61 each have a length (size in the longitudinal direction of the element) of 0.3 mm to 1.0 mm, for example, a width (size in a transverse direction of the element) of 0.5 mm to 30 mm, for example, and a height (size in a thickness direction of the element) of 50 μm to 200 μm, for example. These chambers, however, are not required to have the same size and may have different sizes.

The gas inlet 10 may similarly be formed to penetrate the spacer layer 5 in the thickness direction separately from the first diffusion control part 11. In this case, the first diffusion control part 11 is to be formed inside and adjacent to the gas inlet 10.

The first diffusion control part 11, the second diffusion control part 13, the third diffusion control part 30, and the fourth diffusion control part 60 each include two horizontally long slits. That is to say, they each have openings elongated in a direction perpendicular to the page of the figure in an upper portion and a lower portion in the figure thereof. The slits each have a length (size in the longitudinal direction of the element) of 0.2 mm to 1.0 mm, for example, a width of an opening (size in the transverse direction of the element) of 0.5 mm to 30 mm, for example, and a height of the opening (size in the thickness direction of the element) of 5 μm to 30 μm, for example.

The sensor element 101 includes a reference gas introduction space 43 in the other end portion (a right end portion in the figure) opposite the one end portion in which the gas inlet 10 is provided. The reference gas introduction space 43 is formed between an upper surface 3a of the third substrate 3 and a lower surface 5b of the spacer layer 5. A side portion of the reference gas introduction space 43 is defined by a side surface of the first solid electrolyte layer 4. Oxygen (O₂) and air are introduced into the reference gas introduction space 43 as reference gases, for example.

The gas inlet 10 (first diffusion control part 11) is a part opening to an external space, and the measurement gas is taken from the external space into the sensor element 101 through the gas inlet 10.

The first diffusion control part 11 is a part providing predetermined diffusion resistance to the taken measurement gas.

The buffer space 12 is provided to cancel concentration fluctuations of the measurement gas caused by pressure fluctuations of the measurement gas in the external space. Pulsation of exhaust pressure of the exhaust gas of the vehicle is taken as an example of such pressure fluctuations of the measurement gas, for example.

The second diffusion control part 13 is a part providing predetermined diffusion resistance to the measurement gas introduced from the buffer space 12 into the first chamber 20.

The first chamber 20 is provided as a space to oxidize, when the measurement gas introduced through the second diffusion control part 13 contains a hydrocarbon gas component (an H/C component), the H/C component with oxygen pumped in from the external space. Pumping-in of oxygen is implemented by operation of an adjustment pump cell 21. In the case that the measurement gas is sufficiently lean, however, the adjustment pump cell 21 may pump out oxygen from the first chamber 20.

The adjustment pump cell 21 is an electrochemical pump cell including an inner pump electrode (adjustment electrode) 22, an outer pump electrode (out-of-space pump electrode) 23, and a solid electrolyte present in a portion of the structure 14 sandwiched between these electrodes.

In the adjustment pump cell 21, a voltage Vp0 is applied across the inner pump electrode 22 and the outer pump electrode 23 from a variable power supply 24 disposed outside the sensor element 101 to generate an oxygen pump current (oxygen ion current) Ip0. Oxygen can thereby be pumped into the first chamber 20 from the external space or be pumped out to the external space from the first chamber 20. Assume that a direction of the oxygen pump current Ip0 when oxygen is pumped out from the first chamber 20 is a positive direction of the oxygen pump current Ip0 in the present embodiment.

The inner pump electrode 22 is provided on substantially the entire portions of the lower surface 6b of the second solid electrolyte layer 6 and the upper surface 4a of the first solid electrolyte layer 4 defining the first chamber 20 respectively as a ceiling electrode portion 22a and a bottom electrode portion 22b. The ceiling electrode portion 22a and the bottom electrode portion 22b are connected by an unillustrated conducting portion.

The inner pump electrode 22 is provided as a porous cermet electrode containing at least one of platinum and rhodium (Rh) as a metal component and being rectangular in plan view.

The outer pump electrode 23 is provided, with platinum or an alloy (a Pt—Au alloy) of platinum and gold as a metal component, as a porous cermet electrode containing platinum or the Pt—Au alloy and zirconia and being rectangular in plan view, for example.

In the sensor element 101, the inner pump electrode 22, a reference electrode 42, and a solid electrolyte present in a portion of the structure 14 sandwiched between these electrodes constitute a first chamber sensor cell 80. The first chamber sensor cell 80 is an electrochemical sensor cell to grasp oxygen partial pressure in an atmosphere in the first chamber 20.

The reference electrode 42 is an electrode formed between the first solid electrolyte layer 4 and the third substrate 3 and is provided as a porous cermet electrode containing platinum and zirconia and being rectangular in plan view, for example.

A reference gas introduction layer 48 formed of porous alumina and leading to the reference gas introduction space 43 is provided around the reference electrode 42. A reference gas in the reference gas introduction space 43 is introduced into a surface of the reference electrode 42 via the reference gas introduction layer 48. That is to say, the reference electrode 42 is always in contact with the reference gas.

In the first chamber sensor cell 80, electromotive force (Nernst electromotive force) V0 is generated between the inner pump electrode 22 and the reference electrode 42. The electromotive force V0 has a value in accordance with a difference between an oxygen concentration (oxygen partial pressure) in the first chamber 20 and an oxygen concentration (oxygen partial pressure) of the reference gas. The oxygen concentration (oxygen partial pressure) of the reference gas is basically constant, so that the electromotive force V0 has a value in accordance with the oxygen concentration (oxygen partial pressure) in the first chamber 20.

The third diffusion control part 30 is a part providing predetermined diffusion resistance to the measurement gas introduced from the first chamber 20 into the second chamber 40 and substantially not containing the H/C component.

The second chamber 40 is provided as a space to pump oxygen out of the measurement gas introduced through the third diffusion control part 30 and further reduce (decompose) H₂O and CO₂ contained in the measurement gas as the sensing target gas components to generate hydrogen (H₂) and carbon monoxide (CO), so that the measurement gas does not substantially contain oxygen, H₂O, and CO₂. Pumping-out of oxygen and reduction (decomposition) of H₂O and CO₂ are implemented by operation of a first measurement pump cell 50.

The first measurement pump cell 50 is an electrochemical pump cell including a first measurement electrode 51, the outer pump electrode 23, and a solid electrolyte present in a portion of the structure 14 sandwiched between these electrodes.

In the first measurement pump cell 50, a voltage Vp1 is applied across the first measurement electrode 51 and the outer pump electrode 23 from a variable power supply 52 disposed outside the sensor element 101 to generate an oxygen pump current (oxygen ion current) Ip1. Oxygen can thereby be pumped out to the external space from the second chamber 40. Assume that a direction of the oxygen pump current Ip1 when oxygen is pumped out from the second chamber 40 is a positive direction of the oxygen pump current Ip1 in the present embodiment.

The first measurement electrode 51 is provided on substantially the entire portions of the lower surface 6b of the second solid electrolyte layer 6 and the upper surface 4a of the first solid electrolyte layer 4 defining the second chamber 40 respectively as a ceiling electrode portion 51a and a bottom electrode portion 51b. The ceiling electrode portion 51a and the bottom electrode portion 51b are connected by an unillustrated conducting portion.

The first measurement electrode 51 is provided as a porous cermet electrode containing Pt as a metal component and being rectangular in plan view.

In the sensor element 101, the first measurement electrode 51, the reference electrode 42, and a solid electrolyte present in a portion of the structure 14 sandwiched between these electrodes constitute a second chamber sensor cell 81. The second chamber sensor cell 81 is an electrochemical sensor cell to grasp oxygen partial pressure in an atmosphere in the second chamber 40.

In the second chamber sensor cell 81, electromotive force (Nernst electromotive force) V1 is generated between the first measurement electrode 51 and the reference electrode 42. The electromotive force V1 has a value in accordance with a difference between an oxygen concentration (oxygen partial pressure) in the second chamber 40 and the oxygen concentration (oxygen partial pressure) of the reference gas. Since the oxygen concentration (oxygen partial pressure) of the reference gas is basically constant, the electromotive force V1 has a value in accordance with the oxygen concentration (oxygen partial pressure) in the second chamber 40.

The fourth diffusion control part 60 is a part providing predetermined diffusion resistance to the measurement gas introduced from the second chamber 40 into the third chamber 61 and containing H₂ and CO while substantially not containing H₂O, CO₂, and oxygen.

The third chamber 61 is provided as a space to selectively oxidize all of H₂ from among H₂ and CO contained in the measurement gas introduced through the fourth diffusion control part 60 to generate H₂O again. Generation of H₂O by oxidation of H₂ is implemented by operation of a second measurement pump cell 41.

The second measurement pump cell 41 is an electrochemical pump cell including a second measurement electrode 44, the outer pump electrode 23, and a solid electrolyte present in a portion of the structure 14 sandwiched between these electrodes.

In the second measurement pump cell 41, a voltage Vp2 is applied across the second measurement electrode 44 and the outer pump electrode 23 from a variable power supply 46 disposed outside the sensor element 101 to generate an oxygen pump current (oxygen ion current) Ip2. Oxygen can thereby be pumped into the third chamber 61 from the external space. Assume that a direction of the oxygen pump current Ip2 when oxygen is pumped out from the third chamber 61 is a positive direction of the oxygen pump current Ip2 in the present embodiment.

The second measurement electrode 44 is provided on substantially the entire portion of the upper surface 4a of the first solid electrolyte layer 4 defining the third chamber 61.

The second measurement electrode 44 contains a Pt—Au alloy as a metal component and is provided as a porous cermet electrode containing the Pt—Au alloy and zirconia and being rectangular in plan view, for example. The Pt—Au alloy preferably has an Au concentration of 1 wt % or more and 50 wt % or less and more preferably has an Au concentration of 10 wt % or more and 30 wt % or less. In this case, a selective H₂ oxidation property, that is, a property that, when H₂ and CO coexist in the third chamber 61, only H₂ is selectively oxidized with oxygen pumped in by the second measurement pump cell 41 and CO is not oxidized, of the second measurement electrode 44 is more suitably developed.

In the sensor element 101, the second measurement electrode 44, the reference electrode 42, and a solid electrolyte present in a portion of the structure 14 sandwiched between these electrodes constitute a third chamber sensor cell 82. The third chamber sensor cell 82 is an electrochemical sensor cell to grasp oxygen partial pressure in an atmosphere in the third chamber 61.

In the third chamber sensor cell 82, electromotive force (Nernst electromotive force) V2 is generated between the second measurement electrode 44 and the reference electrode 42. The electromotive force V2 has a value in accordance with a difference between an oxygen concentration (oxygen partial pressure) in the third chamber 61 and the oxygen concentration (oxygen partial pressure) of the reference gas. Since the oxygen concentration (oxygen partial pressure) of the reference gas is basically constant, the electromotive force V2 has a value in accordance with the oxygen concentration (oxygen partial pressure) in the third chamber 61.

The sensor element 101 further includes an electrochemical sensor cell 83 including the outer pump electrode 23, the reference electrode 42, and a solid electrolyte present in a portion of the structure 14 sandwiched between these electrodes. Electromotive force Vref generated between the outer pump electrode 23 and the reference electrode 42 of the sensor cell 83 has a value in accordance with oxygen partial pressure of the measurement gas present outside the sensor element 101.

In addition to the foregoing, the sensor element 101 includes a heater part 70 playing a role in temperature adjustment of heating the sensor element 101 and maintaining the temperature thereof to enhance oxygen ion conductivity of the solid electrolyte forming the structure 14.

The heater part 70 mainly includes a heater electrode 71, a heater element 72, a heater lead 72a, a through hole 73, a heater insulating layer 74, and a heater resistance detection lead, which is not illustrated in FIG. 1. The heater element 72 is hereinafter also simply referred to as a heater 72.

The heater 72 is provided to be sandwiched between the second substrate 2 and the third substrate 3 from below and above and generates heat by being powered from outside through the heater electrode 71 provided on a lower surface 1b of the first substrate 1, the through hole 73, and the heater lead 72a. The heater 72 is buried over the entire region of a range from the buffer space 12 to the third chamber 61 and can heat the sensor element 101 to a predetermined temperature and, further, maintain the temperature.

The heater 72 is provided so that a temperature is highest near the first chamber 20 (near the adjustment electrode 22) and decreases with increasing distance from the first chamber 20 in the longitudinal direction of the element during heating. In the present embodiment, a temperature in a range from the one end portion of the sensor element 101 in which the gas inlet 10 is disposed to the third chamber 61 when the gas sensor 100 is in use (when the sensor element 101 is driven) is referred to as an element driving temperature. The heater 72 performs heating so that the element driving temperature is in a range of 750° C. to 950° C.

The heater insulating layer 74 of alumina and the like is formed above and below the heater 72 to electrically insulate the heater 72 from the second substrate 2 and the third substrate 3. The heater part 70 also includes a pressure dissipation hole 75. The pressure dissipation hole 75 is a part provided to penetrate the third substrate 3 and communicate with the reference gas introduction space 43 and is provided to mitigate a rise in internal pressure associated with a rise in temperature in the heater insulating layer 74.

The gas sensor 100 further includes a controller 110 controlling operation of the sensor element 101 and performing processing to identify concentrations of the sensing target gas components based on currents flowing through the sensor element 101.

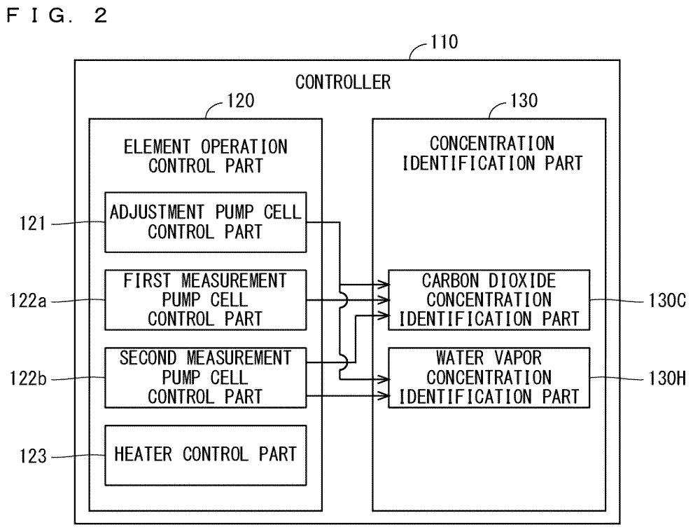

FIG. 2 is a block diagram showing functional components implemented by the controller 110. The controller 110 is configured by one or more electronic circuits including one or more central processing units (CPUs), a storage device, and the like, for example. Each of the electronic circuits is a software functional part implementing a predetermined functional component by a CPU executing a predetermined program stored in the storage device, for example. The controller 110 may naturally be configured by an integrated circuit, such as a field-programmable gate array (FPGA), on which a plurality of electronic circuits are connected in accordance with their functions and the like.

When the gas sensor 100 is attached to the exhaust path of the engine of the vehicle and is used with the exhaust gas flowing along the exhaust path as the measurement gas, some or all of functions of the controller 110 may be implemented by an electronic control unit (ECU) of the vehicle.

The controller 110 includes, as functional components implemented by the CPU executing a predetermined program, an element operation control part 120 controlling operation of each part of the sensor element 101 described above and a concentration identification part 130 performing processing to identify the concentrations of the sensing target gas components contained in the measurement gas.

The element operation control part 120 mainly includes an adjustment pump cell control part 121 controlling operation of the adjustment pump cell 21, a first measurement pump cell control part 122a controlling operation of the first measurement pump cell 50, a second measurement pump cell control part 122b controlling operation of the second measurement pump cell 41, and a heater control part 123 controlling heating operation performed by the heater 72.

On the other hand, the concentration identification part 130 mainly includes a water vapor concentration identification part 130H and a carbon dioxide concentration identification part 130C respectively identifying a concentration of H₂O and a concentration of CO₂ as the main sensing target gas components of the gas sensor 100. The water vapor concentration identification part 130H identifies the concentration of H₂O contained in the measurement gas based on a value of the oxygen pump current Ip0 flowing through the adjustment pump cell 21 acquired by the adjustment pump cell control part 121 and a value of the oxygen pump current Ip2 flowing through the second measurement pump cell 41 acquired by the second measurement pump cell control part 122b.

The carbon dioxide concentration identification part 130C identifies the concentration of CO₂ contained in the measurement gas based on the concentration of H₂O identified by the water vapor concentration identification part 130H (the value of the oxygen pump current Ip2 based on which the concentration of H₂O is identified), the value of the oxygen pump current Ip0 flowing through the adjustment pump cell 21 acquired by the adjustment pump cell control part 121, and a value of the oxygen pump current Ip1 flowing through the first measurement pump cell 50 acquired by the first measurement pump cell control part 122a.

### <Multi-Gas Sensing and Concentration Identification>

A method of sensing a plurality of types of gases (multi-gas sensing) and identifying concentrations of the sensed gases implemented by the gas sensor 100 having a configuration as described above will be described next. Assume hereinafter that the measurement gas is an exhaust gas that essentially contains oxygen, H₂O, and CO₂ and may contain the H/C component.

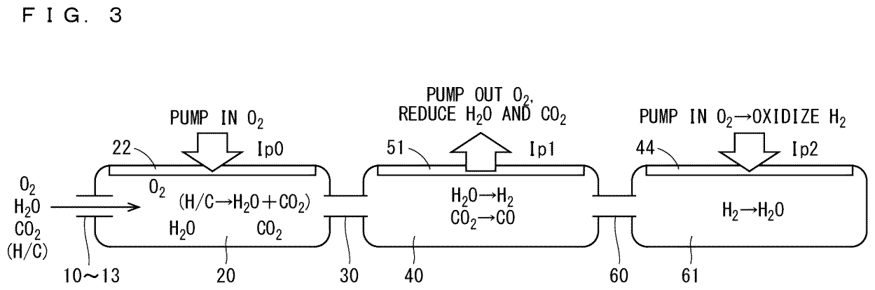

FIG. 3 is a schematic diagram illustrating entry and exit of gases into and from the three chambers (internal spaces) comprised in the sensor element 101 of the gas sensor 100.

First, in the sensor element 101 of the gas sensor 100 according to the present embodiment, the measurement gas is introduced through the gas inlet 10 (first diffusion control part 11), the buffer space 12, and the second diffusion control part 13 into the first chamber 20 as described above. In the first chamber 20, oxygen is pumped in from the external space or is pumped out to the external space to maintain the oxygen concentration (or oxygen partial pressure) constant by operation of the adjustment pump cell 21. Only a case where oxygen is pumped in is illustrated in FIG. 3.

Pumping-in or pumping-out of Oxygen is performed in the way that the adjustment pump cell control part 121 of the controller 110 sets a target value (control voltage) of the electromotive force V0 in the first chamber sensor cell 80 to a value in a range of 400 mV to 700 mV (preferably 400 mV) and feedback-controls the voltage Vp0 applied from the variable power supply 24 to the adjustment pump cell 21 in accordance with a difference between an actual value and the target value of the electromotive force V0 so that the electromotive force V0 is maintained at the target value. A value of the electromotive force V0 significantly deviates from the target value when the measurement gas having a high H/C component content while containing a small amount of oxygen as with a rich atmospheric gas reaches the first chamber 20, and thus the adjustment pump cell control part 121 controls the pump voltage Vp0 applied from the variable power supply 24 to the adjustment pump cell 21 to pump oxygen into the first chamber 20 so that the deviation is reduced.

When the measurement gas contains the H/C component, the adjustment pump cell 21 pumps oxygen into the first chamber 20, so that oxidation of the H/C component to generate H₂O and CO₂ progresses.

On the other hand, when the measurement gas originally does not substantially contain the H/C component, only oxygen is pumped in, essentially.

When a smaller target value of the electromotive force V0 is set, a larger amount of oxygen is pumped in to more surely oxidize the H/C component contained in the measurement gas, but, even if the measurement gas does not contain the H/C component, a large amount of oxygen is pumped in. Pumping-in of the large amount of oxygen is accompanied by an excessive burden on the adjustment pump cell 21, and thus the target value of the electromotive force V0 is only required to be set so that oxygen is pumped in to the extent that substantially all the H/C component is oxidized for practical use. The extent that substantially all the H/C component is oxidized means that the H/C component remaining to the extent that it does not affect accuracy of measurement of the concentrations of H₂O and CO₂ is allowed. In this case, pumping-in of oxygen in a state where the measurement gas does not contain the H/C component is suppressed.

In the case that the measurement gas has a sufficiently large oxygen concentration, however, oxygen may be pumped out depending on the set target value of the electromotive force V0 (depending on the target value of the oxygen concentration or the oxygen partial pressure in the first chamber 20).

In any case, the oxygen partial pressure in the first chamber 20 is maintained at a value in accordance with the target value of the electromotive force V0. It is approximately 10-8 atm when an equation V0=400 mV holds true, for example.

The measurement gas substantially not containing the H/C component is introduced into the second chamber 40. In the second chamber 40, oxygen contained in the measurement gas is pumped out, and H₂O and CO₂ contained in the measurement gas are reduced. That is to say, by operation of the first measurement pump cell 50, oxygen is pumped out of the measurement gas introduced into the second chamber 40, and a reduction (decomposition) reaction (2H₂O→2H₂+O₂ and 2CO₂→2CO+O₂) of H₂O and CO₂ contained in the measurement gas progresses to decompose substantially all H₂O and CO₂ into hydrogen (H₂), carbon monoxide (CO), and oxygen. H₂O contained in the measurement gas herein includes H₂O already contained when being introduced from outside and H₂O generated by oxidation of the H/C component.

Pumping-out of Oxygen and reduction (decomposition) of H₂O and CO₂ are performed in the way that the first measurement pump cell control part 122a of the controller 110 sets a target value (control voltage) of the electromotive force V1 in the second chamber sensor cell 81 to a value in a range of 1000 mV to 1500 mV (preferably 1000 mV) and feedback-controls the voltage Vp1 applied from the variable power supply 52 to the first measurement pump cell 50 in accordance with a difference between an actual value and the target value of the electromotive force V1 so that the electromotive force V1 is maintained at the target value.

The first measurement pump cell 50 operates in this manner, so that oxygen partial pressure in the second chamber 40 is maintained at a much lower value than oxygen partial pressure in the first chamber 20. It is approximately 10-20 atm when an equation V1=1000 mV holds true, for example. The measurement gas thus no longer substantially contains H₂O, CO₂, and oxygen.

The measurement gas containing H₂ and CO while not substantially containing H₂O, CO₂, and oxygen is introduced into the third chamber 61.

In the third chamber 61, oxygen is pumped in by operation of the second measurement pump cell 41, and only H₂ contained in the introduced measurement gas is selectively oxidized.

Pumping-in of Oxygen is performed in the way that the second measurement pump cell control part 122b of the controller 110 sets a target value (control voltage) of the electromotive force V2 in the third chamber sensor cell 82 to a value in a range of 250 mV to 450 mV (preferably 350 mV) and feedback-controls the voltage Vp2 applied from the variable power supply 46 to the second measurement pump cell 41 in accordance with a difference between an actual value and the target value of the electromotive force V2 so that the electromotive force V2 is maintained at the target value.

The second measurement pump cell 41 operates in this manner, so that an oxidation (a combustion) reaction 2H₂+O₂→2H₂O is facilitated, and H₂O in an amount correlating with the total amount of H₂O introduced through the gas inlet 10 and H₂O generated by oxidation of the H/C component in the first chamber 20 is generated again in the third chamber 61. In the present embodiment, H₂O in the correlating amount means that the total amount of H₂O introduced through the gas inlet 10 and H₂O generated by oxidation of the H/C component in the first chamber 20 and the amount of H₂O generated again by oxidation of H₂ generated by decomposition of H₂O are the same or are within a certain error range allowable in terms of measurement accuracy.

The target value of the electromotive force V2 is set to the value in the range of 250 mV to 450 mV, so that oxygen partial pressure in the third chamber 61 is maintained at a value in a range in which almost all H₂ is oxidized but CO is not oxidized. It is approximately 10⁻⁷ atm when an equation V2=350 mV holds true, for example.

Providing the second measurement electrode 44 as the cermet electrode containing the Pt—Au alloy having an Au concentration of 1 wt % or more and 50 wt % or less as the metal component as described above also contributes to improvement in selective H₂ oxidation property.

In the gas sensor in conventional technology, the cermet electrode containing the Pt—Au alloy is provided in the second chamber 40, and the pump cell including the electrode pumps in oxygen for selective oxidation of H₂, but, herein, the first measurement electrode 51 not containing Au as the metal component is provided in the second chamber 40, and the second measurement electrode 44 containing the Pt—Au alloy as the metal component and being responsible for selective oxidation of H₂ is provided to face the third chamber 61 at a lower temperature than the second chamber 40 during operation of the gas sensor 100. Thus, in the gas sensor 100 according to the present embodiment, evaporation of Au from the electrode is suppressed compared with that in the gas sensor in conventional technology.

In addition, any measures to devise a shape (a width and a thickness), placement (a density), and the like of the heater 72 may be taken to further suppress a rise in temperature of the second measurement electrode 44.

In the gas sensor 100 according to the present embodiment operating in the above-mentioned manner, the concentrations of H₂O and CO₂ in the measurement gas are identified based on the oxygen pump current Ip0 flowing through the adjustment pump cell 21 during pumping-in of oxygen for oxidation of the H/C component, the oxygen pump current Ip1 flowing through the first measurement pump cell 50 during pumping-out of oxygen including reduction of H₂O and CO₂, and the oxygen pump current Ip2 flowing through the second measurement pump cell 41 during pumping-in of oxygen for oxidation of H₂.

In identification, however, a contribution of the H/C component to each of the oxygen pump currents Ip0, Ip1, and Ip2 is required to be considered.

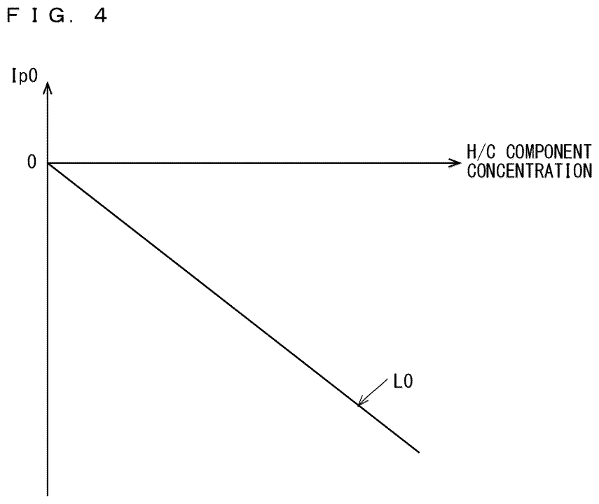

The oxygen pump current Ip0 will be described first. FIG. 4 is a diagram schematically showing dependence of the oxygen pump current Ip0 on the concentration of the H/C component when the measurement gas contains the H/C component.

Although some variation can occur depending on a type of hydrocarbon (more specifically, an atomic ratio of H atoms to C atoms forming hydrocarbon) forming the H/C component, it is regarded that there is a linear relationship as shown by a graph L0 in FIG. 4 between the oxygen pump current Ip0 and the concentration of the H/C component. The oxygen pump current Ip0 monotonically decreases relative to the concentration of the H/C component because the direction of the oxygen pump current Ip0 when oxygen is pumped out is the positive direction.

There is a linear relationship as shown by the graph L0, so that the value of the oxygen pump current Ip0 can be considered to change depending on the concentration of the H/C component changing from moment to moment.

While the graph L0 passes through the origin for ease of understanding in FIG. 4, a point of intersection of the graph L0 and the vertical axis more specifically changes depending on the target value of the electromotive force V0 in the first chamber sensor cell 80.

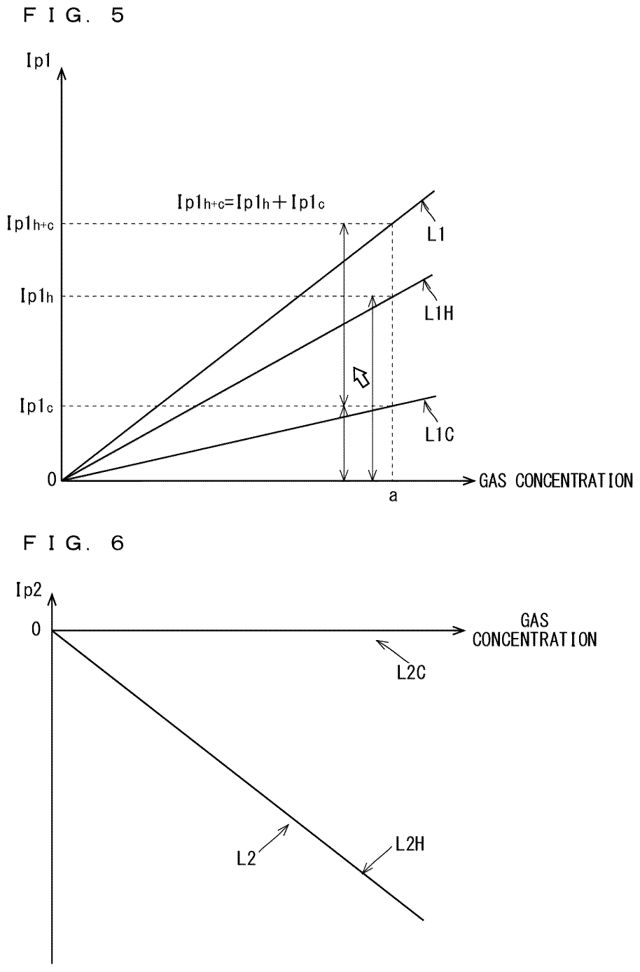

The oxygen pump current Ip1 will be described next. FIG. 5 is a diagram showing an example of dependence of the oxygen pump current Ip1 on gas concentrations in the measurement gas not containing the H/C component. Specifically, FIG. 5 schematically shows, when only one of H₂O and CO₂ alone is contained as a main sensing target gas component and when H₂O and CO₂ having equal concentrations are contained as main sensing target gas components, dependence of the oxygen pump current Ip1 on a concentration of the sensing target gas component and the concentrations of the sensing target gas components.

FIG. 5 shows a graph L1H when H₂O alone is contained as the sensing target gas component, a graph L1C when CO₂ alone is contained as the sensing target gas component, and a graph L1 when H₂O and CO₂ having equal concentrations are contained as the sensing target gas components. These graphs can actually be obtained by operating the gas sensor 100 in an atmosphere of each of model gases containing one or more sensing target gas components having known concentrations and containing oxygen and nitrogen as the balance.

As shown in FIG. 5, the graphs L1H, L1C, and L1 are straight lines increasing monotonically. In addition, a value Ip1_{h+c} of the oxygen pump current Ip1 when the measurement gas equally contains H₂O and CO₂ having any gas concentrations a as the sensing target gas components is the sum of a value of an oxygen pump current Ip1ₕ when H₂O having the gas concentration a alone is contained and a value of an oxygen pump current Ip1c when CO₂ having the gas concentration a alone is contained.

It has also been confirmed that a value of the oxygen pump current Ip1 when H₂O and CO₂ have different proportions is the sum of a value of the oxygen pump current Ip1 when H₂O alone having a concentration in accordance with its proportion is contained and a value of the oxygen pump current Ip1 when CO₂ alone having a concentration in accordance with its proportion is contained, although it is not shown.

That is to say, the following relationship holds true regardless of whether H₂O and CO₂ contained in the measurement gas have equal concentrations or not, where Ip1ᵣ is the value of the oxygen pump current Ip1 when the measurement gas does not contain the H/C component.

$$Ip {1}_{r} = Ip {1}_{h} + Ip {1}_{c} \qquad ( 1 )$$

Next, when the measurement gas contains the H/C component, and the adjustment pump cell 21 pumps oxygen into the first chamber 20, oxygen pumped out from the second chamber 40 by the first measurement pump cell 50 to generate the oxygen pump current Ip1 includes: (a) oxygen originally contained in the measurement gas; (b) oxygen generated by reduction of H₂O and CO₂ originally contained in the measurement gas; (c) oxygen pumped into the first chamber 20 by the adjustment pump cell 21; and (d) oxygen generated when H₂O and CO₂ generated by oxidation of the H/C component contained in the measurement gas upon pumping-in are reduced.

That is to say, when the measurement gas contains the H/C component, the oxygen pump current Ip1 is in accordance with the amount of oxygen (a) to oxygen (d). Oxygen (c) and oxygen (d), however, are oxygen pumped into the first chamber 20 by the adjustment pump cell 21 in the first place. The sum of contributions of oxygen (c) and oxygen (d) in the oxygen pump current Ip1 can thus be considered to be equal to the absolute value |Ip0| of the oxygen pump current Ip0. On the other hand, (a) and (b) correspond to Ip1ᵣ in the equation (1).

That is to say, the following relationship holds true, where Ip1ₜ is a value of the oxygen pump current Ip1 as a whole.

$$Ip {1}_{r} = Ip {1}_{t} - \vert Ip 0 \vert \qquad ( 2 )$$

A value Ip1ᵣ is referred to as an actual reducing current value.

According to the equations (1) and (2), as for the oxygen pump current Ip1 when the measurement gas contains the H/C component, the actual reducing current value Ip1ᵣ obtained by subtracting the absolute value |Ip0| of the oxygen pump current Ip0 from the value Ip1ₜ of the oxygen pump current Ip1 as in the equation (2) can be handled similarly to that when the measurement gas originally contains only oxygen (a) and oxygen (b) and does not contain the H/C component in the relationship indicated by the equation (1).

That is to say, as in a case of the measurement gas not containing H/C, the equation (1) is applicable to the actual reducing current value Ip1ᵣ obtained by subtracting the absolute value of the oxygen pump current Ip0 flowing through the adjustment pump cell 21 from the value of the oxygen pump current Ip1 flowing through the first measurement pump cell 50 obtained by the equation (2).

This means that, by identifying the linear relationship between the oxygen pump current Ip1 and the gas concentrations as shown by the graphs L1H and L1C in advance for H₂O and CO₂ using model gases, the equation (1) and the linear relationship are applicable to the actual reducing current value Ip1ᵣ when the concentrations of H₂O and CO₂ in the measurement gas containing the H/C component are identified.

In the case that the H/C component is not present in the measurement gas, and oxygen is pumped out from the first chamber 20, the following equation simply holds true.

$$Ip {1}_{r} = Ip {1}_{t} \qquad ( 3 )$$

That is to say, when oxygen is pumped out from the first chamber 20, the actual reducing current value Ip1ᵣ is nothing less than the value of the oxygen pump current Ip1 itself. The equation (1) and the above-mentioned linear relationship are also applicable in this case.

The oxygen pump current Ip2 will be described. FIG. 6 is a diagram showing an example of dependence of the oxygen pump current Ip2 on the gas concentrations in the measurement gas not containing the H/C component. Specifically, FIG. 6 schematically shows, when only one of H₂O and CO₂ alone is contained as a main sensing target gas component and when H₂O and CO₂ having equal concentrations are contained as main sensing target gas components, dependence of the oxygen pump current Ip2 on a concentration of the sensing target gas component and the concentrations of the sensing target gas components.

FIG. 6 shows a graph L2H when H₂O alone is contained as the sensing target gas component, a graph L2C when CO₂ alone is contained as the sensing target gas component, and a graph L2 when H₂O and CO₂ having equal concentrations are contained as the sensing target gas components. These graphs can actually be obtained by operating the gas sensor 100 in an atmosphere of each of the model gases containing one or more sensing target gas components having known concentrations and containing oxygen and nitrogen as the balance.

As shown in FIG. 6, the graph L2H of the oxygen pump current Ip2 when only H₂O is contained as the sensing target gas component monotonically decreases (the absolute value thereof monotonically increases). The oxygen pump current Ip2 has a negative value because, the oxygen pump current Ip2 flows in a direction in which oxygen is pumped in to oxidize H₂ generated by reduction in the second chamber 40 again, while the direction of the oxygen pump current when the second measurement pump cell 41 pumps out oxygen is assumed to be the positive direction of the oxygen pump current as described above.

In contrast, the graph L2C of the oxygen pump current Ip2 when only CO₂ is contained as the sensing target gas component is constantly zero regardless of the gas concentration. This corresponds to CO generated by reduction in the second chamber 40 being not oxidized again by operation of the second measurement pump cell 41.

The graph L2 of the oxygen pump current Ip2 when H₂O and CO₂ having equal concentrations are contained as the sensing target gas components coincides with the graph L2H of the oxygen pump current Ip2 when H₂O alone is contained.

It has also been confirmed that a value of the oxygen pump current Ip2 when H₂O and CO₂ have different proportions substantially coincides with the graph L2 of the oxygen pump current Ip2 when H₂O or CO₂ alone having a concentration in accordance with its proportion is contained, although it is not shown.

On the other hand, when the measurement gas contains the H/C component, H₂ and CO flowing into the third chamber 61 include H₂ and CO generated by reduction in the second chamber 40 of H₂O and CO₂ generated by oxidation in the first chamber 20 of the H/C component. Oxygen to oxidize H₂ resulting from the H/C component thus also contributes to the oxygen pump current Ip2 in this case.

That is to say, the following relationship holds true, where Ip2ₜ is a value of the oxygen pump current Ip2 as a whole, Ip2 is a contribution of oxygen to oxidize H₂ resulting from H₂O originally contained in the measurement gas in the oxygen pump current Ip2, Ip2_{HC} is a contribution of oxygen to oxidize H₂ resulting from the H/C component in the oxygen pump current Ip2.

$$Ip {2}_{t} = Ip {2}_{r} + Ip {2}_{H C} \qquad ( 4 )$$

That is to say, the value Ip2ₜ of an actual pump current Ip2 is a value obtained by superimposition of the value Ip2ᵣ and the value Ip2_{HC}. Since the direction of the oxygen pump current Ip2 when oxygen is pumped out is the positive direction, the values Ip2ₜ, Ip2ᵣ, and Ip2_{HC} are each negative.

The value Ip2_{HC} resulting from the H/C component is herein a value obtained by multiplying a predetermined coefficient α (0≤α≤1) indicating an abundance ratio of hydrogen in the H/C component by the value Ip0 of the oxygen pump current Ip0. This is because, while H₂O and CO₂ generated by oxidation of the H/C component are both reduced in the second chamber 40, only H₂ from among H₂ and CO generated by reduction is oxidized again in the third chamber 61.

The equation (4) is thus modified as follows:

$$Ip {2}_{r} = Ip {2}_{t} - α Ip 0 \qquad ( 5 )$$

The coefficient α has a value in accordance with a type of hydrocarbon (more specifically, an atomic ratio of H atoms to C atoms forming hydrocarbon) forming the H/C component contained in the measurement gas and can experimentally be identified in advance by mass spectrometry and the like. A plurality of coefficients α may be identified in accordance with a type and a composition of the H/C component and selectively be used.

A right-hand side of the equation (5) includes only an actual measured value of the pump current and the known coefficient α, so that, by identifying the linear relationship between the oxygen pump current Ip2 and the gas concentrations as shown by the graph L2H for H₂O using model gases, the linear relationship can be applied to the value Ip2ᵣ obtained by substituting the value of the pump current obtained by measurement using the gas sensor 100 into the right-hand side of the equation (5) when the concentrations of H₂O and CO₂ in the measurement gas containing the H/C component are identified.

When the H/C component is not present in the measurement gas, in other words, when Ip0 is positive, the following equation simply holds true.

$$Ip {2}_{r} = Ip {2}_{t} \qquad ( 6 )$$

The above-mentioned linear relationship is also applicable in this case.

In the present embodiment, the concentrations of H₂O and CO₂ in the measurement gas are measured using properties of the oxygen pump current Ip0, the oxygen pump current Ip1, and the oxygen pump current Ip2 as described above. The oxygen pump current Ip0, the oxygen pump current Ip1, and the oxygen pump current Ip2 during actual measurement using the gas sensor 100 are hereinafter also referred to as an H/C oxidation current Ip0, a total reducing current Ip1, and a water vapor equivalent current Ip2.

Specifically, prior to use of the gas sensor 100, characteristics data indicating a relationship between the oxygen pump current Ip1 and a concentration of each gas when the measurement gas contains only one of H₂O and CO₂, does not contain the other one of H₂O and CO₂, and does not contain the H/C component as shown by the graphs L1H and L1C in FIG. 5 (hereinafter also referred to as Ip1-H₂O data and Ip1-CO₂ data) and characteristics data indicating a relationship between the oxygen pump current Ip2 and a concentration of H₂O when the measurement gas contains H₂O and does not contain CO₂ and the H/C component as shown by the graph L2H in FIG. 6 (hereinafter also referred to as Ip2-H₂O data) are acquired in advance using model gases having known concentrations, and these pieces of characteristics data are stored in the controller 110. The Ip1-H₂O data and the Ip1-CO₂ data respectively have a value indicating a contribution of H₂O and a value indicating a contribution of CO₂ in the total reducing current Ip1.

The oxygen pump current Ip1 has a value in accordance with diffusion resistance provided to the measurement gas from the gas inlet 10 to the second chamber 40 of the sensor element 101, and the oxygen pump current Ip2 has a value in accordance with diffusion resistance provided to the measurement gas from the gas inlet 10 to the third chamber 61 of the sensor element 101. The Ip1-H₂O data, the Ip1-CO₂ data, and the Ip2-H₂O data thus strictly vary with each sensor element 101 of the gas sensor 100. These pieces of characteristics data are thus preferably identified for each gas sensor 100. As for gas sensors 100 manufactured under the same condition and from the same lot, however, characteristics data acquired for one particular gas sensor 100 may be applied to another gas sensor 100 from the same lot when it is confirmed that an error is within tolerance.

When actual measurement is performed using the gas sensor 100, the measurement gas is introduced into the sensor element 101 heated to the element driving temperature, and the adjustment pump cell 21, the first measurement pump cell 50, and the second measurement pump cell 41 operate in the above-mentioned manner.

The water vapor concentration identification part 130H acquires the H/C oxidation current Ip0 from the adjustment pump cell control part 121 and acquires the water vapor equivalent current Ip2 from the second measurement pump cell control part 122b. The water vapor concentration identification part 130H calculates the value Ip2ᵣ based on the equation (5) when the H/C oxidation current Ip0 is negative and based on the equation (6) when the H/C oxidation current Ip0 is positive. The concentration of H₂O corresponding to the acquired value is identified based on the Ip2-H₂O data.

Once the concentration of H₂O is identified, the carbon dioxide concentration identification part 130C then acquires the H/C oxidation current Ip0 from the adjustment pump cell control part 121 and acquires the value Ip1ₜ of the total reducing current Ip1 from the first measurement pump cell control part 122a. The actual reducing current value Ip1ᵣ as the value of the pump current obtained by excluding a concentration of the H/C component from the total reducing current Ip1 is calculated based on the equation (2) when the H/C oxidation current Ip0 is negative and based on the equation (3) when the H/C oxidation current Ip0 is positive.

Next, a contribution of H₂O having the concentration identified by the water vapor concentration identification part 130H in the calculated actual reducing current value Ip1ᵣ, that is, the value Ip1ₕ of the current due to reduction of H₂O originally contained in the measurement gas in the total reducing current Ip1 is identified based on the Ip1-H₂O data. The acquired value is subtracted from the actual reducing current value Ip1ᵣ to identify a contribution Ip1c of CO₂ in the total reducing current Ip1. A concentration of CO₂ corresponding to the contribution Ip1c of CO₂ is finally identified based on the Ip1-CO₂ data.

In the gas sensor 100 according to the present embodiment, the concentrations of H₂O and CO₂ in the measurement gas are measured as described above.

As described above, in the gas sensor according to the present embodiment, even when the measurement gas containing H₂O and CO₂ contains the H/C component as with a rich atmospheric gas, the concentrations of H₂O and CO₂ can be measured while the influence of the H/C component is eliminated.

In addition, in a case of the gas sensor according to the present embodiment, H₂O and CO₂ are not reduced in the first chamber to be at a highest temperature during operation in contrast to the gas sensor in conventional technology, so that a voltage applied to the adjustment pump cell pumping in or pumping out oxygen between the first chamber and the outside is suppressed to be lower than that in the gas sensor in conventional technology in which oxygen is pumped out from the first chamber, thereby to suitably suppress cracking and blackening of the sensor element.

An electrode disposed in a chamber as an electrode containing a Pt—Au alloy as a metal component is only the second measurement electrode provided in the third chamber, and the electrode containing the Pt—Au alloy is not provided in the first chamber and the second chamber to be at a higher temperature than the third chamber, so that evaporation of Au from the electrode is suppressed compared with that in conventional technology.

That is to say, according to the present embodiment, a multi-gas sensor with more long-term reliability than before is implemented.

### Modifications

In the gas sensor 100 according to the above-mentioned embodiment, the sensor element 101 includes the gas distribution part including the first chamber 20, the second chamber 40, and the third chamber 61 communicating via the diffusion control parts. With respect to the measurement gas introduced sequentially into the chambers of the gas distribution part under predetermined diffusion resistance, the adjustment pump cell 21 pumps in or pumps out oxygen and the H/C component is oxidized with oxygen as pumped in when the measurement gas contains the H/C component in the first chamber 20, the first measurement pump cell 50 pumps out oxygen to reduce H₂O and CO₂ in the second chamber 40, and the second measurement pump cell 41 is used to selectively oxidize H₂ generated by reduction of H₂O in the third chamber 61. As a result, the concentrations of H₂O and CO₂ in the measurement gas are measured based on the magnitudes of the currents flowing through the respective pump cells.

Such measurement using the gas sensor 100 is considered to be implemented by suppression of a flow of the measurement gas from outside the element into the first chamber 20 using the first diffusion control part 11 and the second diffusion control part 13, suppression of a flow of the measurement gas in which oxygen remains from the first chamber 20 into the second chamber 40 using the third diffusion control part 30, and further suppression of a flow of the measurement gas in which H₂O and CO₂ remain from the second chamber 40 into the third chamber 61 using the fourth diffusion control part 60. That is to say, it can be said that the measurement gas reaching the inner pump electrode 22 of the adjustment pump cell 21, the first measurement electrode 51 of the first measurement pump cell 50, and the second measurement electrode 44 of the second measurement pump cell 41 is suitably controlled by the respective diffusion control parts so that gases not targeted for operation in the respective pump cells do not reach the respective electrodes, thereby to enable multi-gas sensing using the gas sensor 100.

Viewed another way, this means that a configuration different from the configuration of the gas distribution part of the sensor element 101 can be adopted as long as pumping-in or pumping-out of oxygen using the adjustment pump cell 21, reduction of H₂O and CO₂ using the first measurement pump cell 50, and selective oxidation of H₂ generated by reduction of H₂O using the second measurement pump cell 41 with respect to the measurement gas reaching the inner pump electrode 22, the first measurement electrode 51, and the second measurement electrode 44 are performed successfully in view of securing measurement accuracy. For example, even a configuration in which the three chambers communicating via the diffusion control parts are not included can be adopted to implement multi-gas sensing.

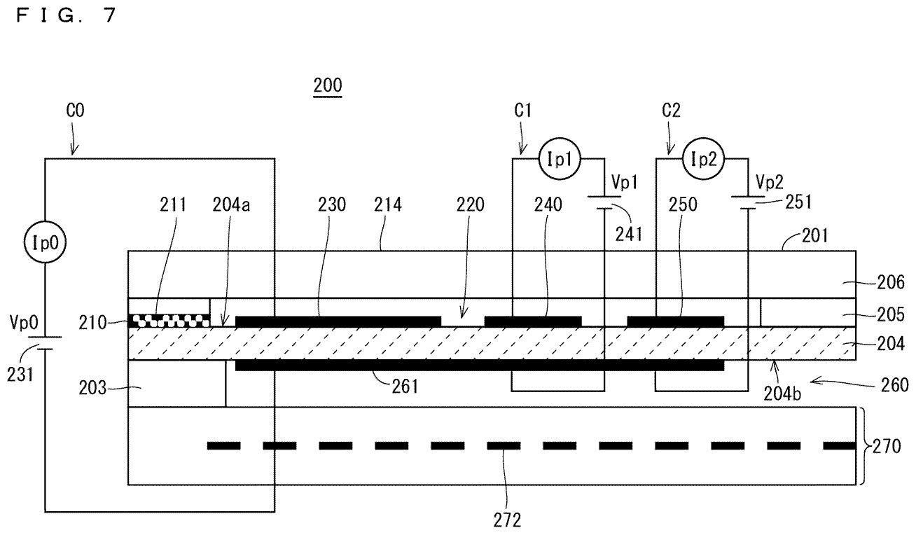

FIG. 7 is a diagram schematically showing one example of a configuration of a gas sensor 200 according to a modification with the foregoing in mind. The gas sensor 200 is a multi-gas sensor sensing a plurality of types of gas components and measuring concentrations thereof using a sensor element 201. In the gas sensor 200, control performed by the controller 110 enables multi-gas sensing with at least water vapor (H₂O) and carbon dioxide (CO₂) as main sensing target gas components as described below as in the gas sensor 100. FIG. 7 includes a vertical cross-sectional view taken along a longitudinal direction of the sensor element 201.

The sensor element 201 is an elongated planar structure including a laminate of a sensor part 214 and a heater part 270.

The sensor part 214 is formed by laminating a plurality of substrate layers of ceramics. Specifically, the sensor part 214 has a configuration in which four layers including a first substrate 203, a second substrate 204, a third substrate 205, and a fourth substrate 206 are sequentially laminated from the bottom. From among them, at least the second substrate 204 is formed of an oxygen-ion conductive solid electrolyte, such as zirconia. The first substrate 203, the third substrate 205, and the fourth substrate 206 may be formed of a solid electrolyte or may be formed of an insulating material, such as alumina. In the sensor part 214, the first substrate 203 is adjacent to the heater part 270.

A gas inlet 210 through which a measurement gas is introduced is provided in one end portion (a left end portion in the figure) of the sensor part 214. More specifically, a diffusion control part 211 formed of a porous body having a porosity of approximately 10% to 50% is buried in one end portion of the third substrate 205, and an exposed portion in one end portion of the diffusion control part 211 is the gas inlet 210. The diffusion control part 211 has a length (size in the longitudinal direction of the element) of 0.5 mm to 1.0 mm, for example, a width (size in a transverse direction of the element) of 1.5 mm to 3 mm, for example, and a height (size in a thickness direction of the element) of 10 μm to 20 μm, for example.

The sensor part 214 also includes a single internal chamber 220 adjacent to the diffusion control part 211. The internal chamber 220 is formed to penetrate the third substrate 205 in the thickness direction. The internal chamber 220 has a length (size in the longitudinal direction of the element) of 6.0 mm to 12.0 mm, for example, a width (size in the transverse direction of the element) of 1.5 mm to 2.5 mm, for example, and a height (size in the thickness direction of the element) of 50 μm to 200 μm, for example.

That is to say, it can be said that the diffusion control part 211 and the internal chamber 220 constitute the gas distribution part communicating with the gas inlet 210 in the sensor element 201.

An adjustment electrode 230, a first measurement electrode 240, and a second measurement electrode 250 are arranged on an exposed surface 204a of the second substrate 204 to the internal chamber 220 in order of proximity to the gas inlet 210 on a left side in the figure to face the internal chamber 220 while being spaced apart at predetermined intervals. They are provided as porous cermet electrodes similar to the inner pump electrode (adjustment electrode) 22, the first measurement electrode 51, and the second measurement electrode 44 of the sensor element 101.

The sensor part 214 further includes a reference gas introduction space 260 opening in the other end portion of the sensor element 201. The reference gas introduction space 260 is formed to penetrate the first substrate 203 in the thickness direction. Oxygen (O₂) and air are introduced into the reference gas introduction space 260 as reference gases, for example.

A reference electrode 261 is provided on an exposed surface 204b of the second substrate 204 to the reference gas introduction space 260. The reference electrode 261 is preferably provided over an entire range of arrangement of the adjustment electrode 230, the first measurement electrode 240, and the second measurement electrode 250 provided on the exposed surface 204a opposite the exposed surface 204b. The reference electrode 261 is provided as a porous cermet electrode containing platinum and zirconia and being rectangular in plan view, for example.

As with the heater part 70 of the sensor element 101, the heater part 270 is configured to heat the sensor element 201 to a predetermined temperature and, further, maintain the temperature by powering of the heater element 272 (also simply referred to as a heater 272) from outside the element. A configuration similar to the configuration of the heater part 70 of the sensor element 101 is applicable to the heater part 270. Alternatively, a configuration in which the heater element 272 is buried in an insulator may be used.

The heater 272 is provided so that a temperature is highest near the adjustment electrode 230 and decreases with increasing distance from the adjustment electrode 230 in the longitudinal direction of the element during heating.

In addition, the sensor element 201 includes an adjustment pump cell C0, a first measurement pump cell C1, and a second measurement pump cell C2.

The adjustment pump cell C0 is an electrochemical pump cell including the adjustment electrode 230, the reference electrode 261, and the second substrate 204 sandwiched between these electrodes. In the adjustment pump cell C0, the voltage Vp0 is applied across the adjustment electrode 230 and the reference electrode 261 from a variable power supply 231 disposed outside the sensor element 201 to generate the oxygen pump current (oxygen ion current) Ip0. Operation of the adjustment pump cell C0 is controlled by the adjustment pump cell control part 121 of the controller 110.

The first measurement pump cell C1 is an electrochemical pump cell including the first measurement electrode 240, the reference electrode 261, and the second substrate 204 sandwiched between these electrodes. In the first measurement pump cell C1, the voltage Vp1 is applied across the first measurement electrode 240 and the reference electrode 261 from a variable power supply 241 disposed outside the sensor element 201 to generate the oxygen pump current (oxygen ion current) Ip1. Operation of the first measurement pump cell C1 is controlled by the first measurement pump cell control part 122a of the controller 110.

The second measurement pump cell C2 is an electrochemical pump cell including the second measurement electrode 250, the reference electrode 261, and the second substrate 204 sandwiched between these electrodes. In the second measurement pump cell C2, the voltage Vp2 is applied across the second measurement electrode 250 and the reference electrode 261 from a variable power supply 251 disposed outside the sensor element 201 to generate the oxygen pump current (oxygen ion current) Ip2. Operation of the second measurement pump cell C2 is controlled by the second measurement pump cell control part 122b of the controller 110.

As described above, in the sensor element 201, the adjustment electrode 230, the first measurement electrode 240, and the second measurement electrode 250 are arranged in the single internal chamber 220 in contrast to the sensor element 101 of the gas sensor 100. Nevertheless, the diffusion control part 211 and the internal chamber 220 are provided on a condition as described above to suitably provide diffusion resistance to the measurement gas introduced into the internal chamber 220, in other words, to suitably control a flow rate of the measurement gas, and therefore, multi-gas sensing with at least water vapor (H₂O) and carbon dioxide (CO₂) as the main sensing target gas components while eliminating the influence of the H/C component even when the measurement gas contains the H/C component is enabled also in the gas sensor 200 including the sensor element 201 under control performed by the controller 110 as in the gas sensor 100.

Specifically, the measurement gas introduced from the gas inlet 210 into the internal chamber 220 through the diffusion control part 211 sequentially reaches the adjustment electrode 230, the first measurement electrode 240, and the second measurement electrode 250. The adjustment pump cell C0 performs operation to pump in or pump out oxygen between the first chamber 20 and the outside so that the oxygen concentration in the first chamber 20 is maintained constant. Thus, when the measurement gas contains the H/C component, the H/C component is oxidized to generate H₂O and CO₂. The first measurement pump cell C1 performs operation to pump out oxygen to reduce H₂O and CO₂ contained in the measurement gas having reached the first measurement electrode 240. The second measurement pump cell C2 performs operation to pump in oxygen to selectively oxidize H₂ generated by reduction of H₂O performed by the first measurement pump cell C1 and having reached the second measurement electrode 250.

In this case, the measurement gas flows at a flow rate at which the measurement gas in which the H/C component remains does not pass through the adjustment electrode 230 and reach the first measurement electrode 240 and the measurement gas in which H₂O and CO₂ remain does not pass through the first measurement electrode 240 and reach the second measurement electrode 250, so that a current flowing through each pump cell is equivalent to a current flowing through each pump cell of the gas sensor 100. Thus, in the gas sensor 200, the water vapor concentration identification part 130H and the carbon dioxide concentration identification part 130C can accurately identify the concentrations of H₂O and CO₂ in the measurement gas as in the gas sensor 100.

What is claimed is:

1. A gas sensor capable of measuring concentrations of a plurality of sensing target gas components contained in a measurement gas, the measurement gas at least containing water vapor and carbon dioxide, the gas sensor comprising:
a sensor element having a structure formed of an oxygen-ion conductive solid electrolyte; and
a controller controlling operation of the gas sensor, wherein
the sensor element comprises:
a gas inlet through which the measurement gas is introduced;
an internal chamber communicating with the gas inlet via a diffusion control part;
an adjustment electrode, a first measurement electrode, and a second measurement electrode each provided to face the internal chamber and arranged in order of proximity to the gas inlet while being spaced apart at predetermined intervals;
an adjustment pump cell including the adjustment electrode, an out-of-space pump electrode provided at a location other than a location in the internal chamber, and a portion of the solid electrolyte present between the adjustment electrode and the out-of-space pump electrode;
a first measurement pump cell including the first measurement electrode, the out-of-space pump electrode, and a portion of the solid electrolyte present between the first measurement electrode and the out-of-space pump electrode;
a second measurement pump cell including the second measurement electrode, the out-of-space pump electrode, and a portion of the solid electrolyte present between the second measurement electrode and the out-of-space pump electrode; and
a heater heating the sensor element,
the adjustment pump cell pumps oxygen into the internal chamber from an external space so that, when the measurement gas having reached the adjustment electrode contains a hydrocarbon gas component, the hydrocarbon gas component is oxidized,
the first measurement pump cell pumps oxygen out of the measurement gas having reached the first measurement electrode so that substantially all water vapor and carbon dioxide contained in the measurement gas are reduced,
the second measurement pump cell pumps oxygen into the internal chamber to selectively oxidize hydrogen generated by reduction of water vapor and contained in the measurement gas having reached the second measurement electrode, and
the controller identifies:
a concentration of water vapor contained in the measurement gas based on a value of a hydrocarbon equivalent current as an oxygen pump current flowing between the adjustment electrode and the out-of-space pump electrode when the hydrocarbon gas component is oxidized with oxygen pumped in by the adjustment pump cell and a value of a water vapor equivalent current as an oxygen pump current flowing between the second measurement electrode and the out-of-space pump electrode when hydrogen is oxidized with oxygen pumped in by the second measurement pump cell; and
a concentration of carbon dioxide contained in the measurement gas based on the value of the hydrocarbon equivalent current, the value of the water vapor equivalent current, and a value of a total reducing current as an oxygen pump current flowing between the first measurement electrode and the out-of-space pump electrode when water vapor and carbon dioxide are reduced by the first measurement pump cell pumping out oxygen.

2. The gas sensor according to claim 1, wherein
the internal chamber includes a first chamber, a second chamber, and a third chamber communicating sequentially in order of proximity to the gas inlet via different diffusion control parts,
the adjustment electrode is disposed in the first chamber,
the first measurement electrode is disposed in the second chamber, and
the second measurement electrode is disposed in the third chamber.

3. The gas sensor according to claim 2, wherein
the controller stores:
Ip1-H₂O data indicating a relationship between an oxygen pump current flowing through the first measurement pump cell and a concentration of carbon dioxide when the measurement gas contains water vapor and does not contain a hydrocarbon gas and carbon dioxide, the Ip1-H₂O data being identified in advance;
Ip1-CO₂ data indicating a relationship between an oxygen pump current flowing through the first measurement pump cell and a concentration of water vapor when the measurement gas contains carbon dioxide and does not contain the hydrocarbon gas and water vapor, the Ip1-CO₂ data being identified in advance;
Ip2-H₂O data indicating a relationship between an oxygen pump current flowing through the second measurement pump cell and a concentration of water vapor when the measurement gas contains water vapor and does not contain the hydrocarbon gas and carbon dioxide, the Ip2-H₂O data being identified in advance; and
a coefficient indicating an abundance ratio of hydrogen to the hydrocarbon gas component contained in the measurement gas, the coefficient being identified in advance,
calculates a first difference value as a difference between the water vapor equivalent current and the product of the hydrocarbon equivalent current and the coefficient and identifies a concentration of water vapor corresponding to the first difference value in the Ip2-H₂O data as the concentration of water vapor contained in the measurement gas, and
identifies:
a contribution of water vapor by reduction in the total reducing current based on the identified concentration of water vapor contained in the measurement gas and the Ip1-H₂O data,
an actual reducing current value as a value of a current flowing upon reduction of water vapor and carbon dioxide originally contained in the measurement gas in the total reducing current and then subtracts the contribution from the actual reducing current value to calculate a second difference value, and
a concentration of carbon dioxide corresponding to the second difference value in the Ip1-CO₂ data as the concentration of carbon dioxide contained in the measurement gas.

4. The gas sensor according to claim 2, wherein
the second measurement electrode is a cermet electrode containing a Pt—Au alloy as a metal component, and the Pt—Au alloy has an Au concentration of 1 wt % or more and 50 wt % or less.

5. The gas sensor according to claim 4, wherein
the adjustment electrode and the first measurement electrode are cermet electrodes containing Pt and not containing Au.

6. The gas sensor according to claim 2, wherein
the heater heats the sensor element so that a temperature is highest near the adjustment electrode in the internal chamber and decreases with increasing distance from the adjustment electrode in a longitudinal direction of the sensor element.

7. A concentration measurement method of measuring concentrations of a plurality of sensing target gas components contained in a measurement gas using a gas sensor, the measurement gas at least containing water vapor and carbon dioxide, wherein
the gas sensor includes a sensor element having an elongated planar structure formed of an oxygen-ion conductive solid electrolyte,
the sensor element comprises:
a gas inlet through which the measurement gas is introduced;
an internal chamber communicating with the gas inlet via a diffusion control part;
an adjustment electrode, a first measurement electrode, and a second measurement electrode each provided to face the internal chamber and arranged in order of proximity to the gas inlet while being spaced apart at predetermined intervals;
an adjustment pump cell including the adjustment electrode, an out-of-space pump electrode provided at a location other than a location in the internal chamber, and a portion of the solid electrolyte present between the adjustment electrode and the out-of-space pump electrode;
a first measurement pump cell including the first measurement electrode, the out-of-space pump electrode, and a portion of the solid electrolyte present between the first measurement electrode and the out-of-space pump electrode;
a second measurement pump cell including the second measurement electrode, the out-of-space pump electrode, and a portion of the solid electrolyte present between the second measurement electrode and the out-of-space pump electrode; and
a heater heating the sensor element, and
the concentration measurement method using the gas sensor comprises:
a) pumping, using the adjustment pump cell, oxygen into the internal chamber from an external space so that, when the measurement gas having reached the adjustment electrode contains a hydrocarbon gas component, the hydrocarbon gas component is oxidized;
b) pumping, using the first measurement pump cell, oxygen out of the measurement gas having reached the first measurement electrode so that substantially all water vapor and carbon dioxide contained in the measurement gas having reached the first measurement electrode are reduced;
c) pumping, using the second measurement pump cell, oxygen into the internal chamber to selectively oxidize hydrogen generated by reduction of water vapor and contained in the measurement gas having reached the second measurement electrode;
d) identifying a concentration of water vapor contained in the measurement gas based on a value of a hydrocarbon equivalent current as an oxygen pump current flowing between the adjustment electrode and the out-of-space pump electrode when the hydrocarbon gas component is oxidized with oxygen pumped in using the adjustment pump cell and a value of a water vapor equivalent current as an oxygen pump current flowing between the second measurement electrode and the out-of-space pump electrode when hydrogen is oxidized with oxygen pumped in using the second measurement pump cell; and
e) identifying a concentration of carbon dioxide contained in the measurement gas based on the value of the hydrocarbon equivalent current, the value of the water vapor equivalent current, and a value of a total reducing current as an oxygen pump current flowing between the first measurement electrode and the out-of-space pump electrode when water vapor and carbon dioxide are reduced by pumping out oxygen using the first measurement pump cell.

8. The concentration measurement method using the gas sensor according to claim 7, wherein
the internal chamber includes a first chamber, a second chamber, and a third chamber communicating sequentially in order of proximity to the gas inlet via different diffusion control parts,
the adjustment electrode is disposed in the first chamber,
the first measurement electrode is disposed in the second chamber, and
the second measurement electrode is disposed in the third chamber.

9. The concentration measurement method using the gas sensor according to claim 8, further comprising
f) prior to the steps a) to e), identifying in advance:
Ip1-H₂O data indicating a relationship between an oxygen pump current flowing through the first measurement pump cell and a concentration of water vapor when the measurement gas contains water vapor and does not contain carbon dioxide;
Ip1-CO₂ data indicating a relationship between an oxygen pump current flowing through the first measurement pump cell and a concentration of carbon dioxide when the measurement gas contains carbon dioxide and does not contain water vapor;
Ip2-H₂O data indicating a relationship between an oxygen pump current flowing through the second measurement pump cell and a concentration of water vapor when the measurement gas contains water vapor and does not contain carbon dioxide; and
a coefficient indicating an abundance ratio of hydrogen to the hydrocarbon gas component contained in the measurement gas, wherein
in the step d), a first difference value as a difference between the water vapor equivalent current and the product of the hydrocarbon equivalent current and the coefficient is calculated, and then a concentration of water vapor corresponding to the first difference value in the Ip2-H₂O data is identified as the concentration of water vapor contained in the measurement gas, and
in the step e), a contribution of water vapor by reduction in the total reducing current is identified based on the concentration of water vapor contained in the measurement gas identified in the step d) and the Ip1-H₂O data, an actual reducing current value as a value of a current flowing upon reduction of water vapor and carbon dioxide originally contained in the measurement gas in the total reducing current is identified and then the contribution is subtracted from the actual reducing current value to calculate a second difference value, and a concentration of carbon dioxide corresponding to the second difference value in the Ip1-CO₂ data is identified as the concentration of carbon dioxide contained in the measurement gas.

10. The concentration measurement method using the gas sensor according to claim 8, wherein
the second measurement electrode is a cermet electrode containing a Pt—Au alloy as a metal component, and the Pt—Au alloy has an Au concentration of 1 wt % or more and 50 wt % or less.

11. The concentration measurement method using the gas sensor according to claim 10, wherein
the adjustment electrode and the first measurement electrode are cermet electrodes containing Pt and not containing Au.

12. The concentration measurement method using the gas sensor according to claim 8, wherein
the heater heats the sensor element so that a temperature is highest near the adjustment electrode in the internal chamber and decreases with increasing distance from the adjustment electrode in a longitudinal direction of the sensor element.

13. The gas sensor according to claim 3, wherein
the second measurement electrode is a cermet electrode containing a Pt—Au alloy as a metal component, and the Pt—Au alloy has an Au concentration of 1 wt % or more and 50 wt % or less.

14. The gas sensor according to claim 13, wherein
the adjustment electrode and the first measurement electrode are cermet electrodes containing Pt and not containing Au.

15. The gas sensor according to claim 3, wherein
the heater heats the sensor element so that a temperature is highest near the adjustment electrode in the internal chamber and decreases with increasing distance from the adjustment electrode in a longitudinal direction of the sensor element.

16. The gas sensor according to claim 7, wherein
the heater heats the sensor element so that a temperature is highest near the adjustment electrode in the internal chamber and decreases with increasing distance from the adjustment electrode in a longitudinal direction of the sensor element.

17. The concentration measurement method using the gas sensor according to claim 9, wherein
the second measurement electrode is a cermet electrode containing a Pt—Au alloy as a metal component, and the Pt—Au alloy has an Au concentration of 1 wt % or more and 50 wt % or less.

18. The concentration measurement method using the gas sensor according to claim 17, wherein
the adjustment electrode and the first measurement electrode are cermet electrodes containing Pt and not containing Au.

19. The concentration measurement method using the gas sensor according to claim 9, wherein
the heater heats the sensor element so that a temperature is highest near the adjustment electrode in the internal chamber and decreases with increasing distance from the adjustment electrode in a longitudinal direction of the sensor element.

20. The concentration measurement method using the gas sensor according to claim 10, wherein
the heater heats the sensor element so that a temperature is highest near the adjustment electrode in the internal chamber and decreases with increasing distance from the adjustment electrode in a longitudinal direction of the sensor element.
